(19) United States
(12) Patent Application Publication
(10) Pub. No.: US 2024/0424064 A1
(43) Pub. Date: Dec. 26, 2024

(54) INSULIN CONTAINING PHARMACEUTICAL COMPOSITIONS

(71) Applicant: Novo Nordisk A/S, Bagsvaerd (DK)

(72) Inventors: Mathias Norrman, Staffanstorp (SE); Susanne Hostrup, Vaerloese (DK); Dorte Bjerre Steensgaard, Maaloev (DK); Holger Martin Strauss, Frederiksberg (DK); Rosa Rebecca Erritzoee Hansen, Koebenhavn OE (DK); Svend Havelund, Bagsvaerd (DK); Morten Schlein, Vaerloese (DK); Jesper Soendergaard Pedersen, Oelstykke (DK)

(21) Appl. No.: 18/823,969

(22) Filed: Sep. 4, 2024

Related U.S. Application Data
(63) Continuation of application No. 17/692,238, filed on Mar. 11, 2022.
Application No. 17/692,238 is a continuation of application No. 16/984,990, filed on Aug. 4, 2020, now abandoned.
Application No. 16/984,990 is a continuation of application No. 15/843,016, filed on Dec. 15, 2017, now abandoned.

(30) Foreign Application Priority Data
Dec. 16, 2016 (EP) 16204688.2

(51) Int. Cl. A61K 38/28; A61K 47/02; A61K 47/10; A61P 5/48

(52) U.S. Cl. CPC A61K 38/28; A61K 47/02; A61K 47/10; A61P 5/48

(57) ABSTRACT

The present invention is in the field of pharmaceutical compositions for the treatment of medical conditions relating to diabetes. More specifically the invention provides pharmaceutical compositions comprising a long-acting acylated derivative of a human insulin analogue, and to the medical use of such compositions for basal insulin administration therapy.

### CROSS-REFERENCE TO RELATED APPLICATIONS

This application is a continuation of U.S. application Ser. No. 17/692,238, filed Mar. 11, 2022, which is a continuation of U.S. application Ser. No. 16/984,990, filed Aug. 4, 2020 (now abandoned), which is a continuation of U.S. application Ser. No. 15/843,016, filed Dec. 15, 2017 (now abandoned), which claims priority to European Patent Application 16204688.2, filed Dec. 16, 2016; the contents of which are incorporated herein by reference.

### TECHNICAL FIELD

The present invention is in the field of pharmaceutical compositions for the treatment of medical conditions relating to diabetes. More specifically the invention provides pharmaceutical compositions comprising a long-acting acylated derivative of a human insulin analogue, and to the medical use of such compositions for basal insulin administration therapy.

### BACKGROUND ART

The primary objective of insulin therapy in the treatment of metabolic disorders is to produce sustained near-normal glycemia by replacing or supplementing endogenous insulin secretion in as physiological a manner as possible, post-prandially as well as between meals and overnight. Separating basal and meal-related (bolus) insulin requirements represents a systematic approach to subcutaneous insulin therapy.

The most relevant pharmacological characteristics of any given insulin—onset of action, peak effect profile, duration of action, etc.—are largely determined by its absorption kinetics from the subcutaneous injection site into the systemic circulation.

Non-covalent zinc mediated oligomerisation is a well-described property of insulin products. Under physiological pH, human insulin is soluble but has a tendency to self-associate into well-defined hexamers (i.e. a unit of six insulin molecules), by coordination of two zinc ions (Zn⁺⁺) to high affinity (B10His) binding sites. It is also well-known that phenolic ligands, especially phenol, bind specifically to the insulin hexamer and promote R-state hexamer formation. Upon injection, phenolic ligands diffuses quickly from the injection site. In the absence of phenol at the injection site, the conformation and size of the insulin oligomer may change, as well as the viscosity of the insulin-containing solution, contributing to a prolonged action profile.

Jonassen et al, Pharm. Res. 2012 29 2104-2114 describe the protraction mechanism of insulin degludec, an insulin derivative with acylated fatty di-acid chain for once-daily administration, and describe the correlation between high zinc concentration and the protraction. WO 2009/115469 describes various long-acting insulin derivatives with acylated fatty di-acid chains. WO 2013/153000 describes the formulation of those long-acting insulin derivatives for subcutaneous administration, which contains high level of zinc (no less than 3.5 Zn⁺⁺/six moles of insulin derivatives). It was designed in order to obtain the prolonged duration of action commensurate with a once-weekly administration profile. High content of Zn⁺⁺ in the formulations described in WO 2009/115469 lead to a prolonged PK profile.

WO 2009/063072 discloses pharmaceutical compositions for parenteral administration comprising a basal insulin derivative (e.g. degludec) and a GLP-1 derivative (e.g, liraglutid). Because liraglutide monomer binds with zinc to form di-heptmer, zinc level higher than the degludec mono formulation is necessary in the combo formulation, to achieve comparable PK profile of degludec, and achieve acceptable physical stability.

### SUMMARY OF THE INVENTION

According to the present invention a new formulation of the long-acting insulin derivatives has been developed, which is capable of promoting a conformational state and oligomerisation pattern more closely resembling that of human insulin, i.e. hexamers, especially R6 hexamers.

In another aspect, the invention provides a pharmaceutical composition comprising a selected long-acting insulin derivative in a unique combination of excipients carefully formulated in order to reduce formation of oligomers at the injection site, while still showing PK/PD properties suited for a once-weekly administration.

In another aspect, the invention provides a pharmaceutical composition with decreased viscosity upon injection and accordingly decreased propensity to create any discomfort upon injection.

In another aspect, the invention provides a pharmaceutical composition with improved stability.

In another aspect, the invention provides a pharmaceutical composition for use as a medicament for the treatment of a metabolic disorder.

Other objects of the invention will be apparent to the person skilled in the art from the following detailed description and examples.

### DETAILED DISCLOSURE OF THE INVENTION

In its first aspect the invention provides pharmaceutical composition comprising an insulin derivative selected from the group consisting of
A14E, B16H, B25H, B29K((N^{ϵ}-Eicosanedioyl-γGlu-[2-(2-{2-[2-(2-aminoethoxy)-ethoxy]acetylamino}ethoxy)ethoxy]acetyl)), desB30 human insulin; (Compound 1);
A14E, B16H, B25H, B29K(N^{ϵ}-Hexadecandioyl-γGlu), desB30 human insulin (Compound 2);
A14E, B16H, B25H, B29K(N^{ϵ}-Eicosanedioyl-γGlu), desB30 human insulin (Compound 3); and
A14E, B25H, desB27, B29K(N^{ϵ}-Octadecandioyl-γGlu), desB30 human insulin (Compound 4); and further comprising of from about 1 to about 2% (weight/weight) of glycerol; of from about 45 to about 75 mM of phenol; of from about 0 to about 19 mM of m-cresol; of from about 1.5 to about 2.5 moles of zinc ions per six moles of said insulin derivative; not more than about 75 mM of sodium chloride; and having a pH value in the range of from 7.2 to 8.0.

In anther aspect, the invention provides pharmaceutical compositions further comprising an insulinotropic GLP-1 compound, and in particular the insulinotropic GLP-1 compound known as semaglutide.

Semaglutide may be described by the structure Aib8, Lys26 (OEG-OEG-gamma-Glu-C18-diacid),Arg34) GLP-1 H(7-37)-OH, which may also be designated as (N-epsilon26-[2-(2-{2-[2-(2-{2-[(S)-4-Carboxy-4-(17-carboxyheptadecanoylamino) butyrylamino]ethoxy}ethoxy)acetylamino]ethoxy}ethoxy)-acetyl][Aib8,Arg34]GLP-1-(7-37), c.f. disclosure in WO 2006/097537.

The present invention may be further characterised by reference to one or more of the following features or embodiments:
1. A pharmaceutical composition of the invention comprising an insulin derivative, which is A14E, B16H, B25H, B29K((N^{ϵ}-Eicosanedioyl-γGlu-[2-(2-{2-[2-(2-aminoethoxy) ethoxy] acetylamino} ethoxy) ethoxy] acetyl)), desB30 human insulin (Compound 1).
2. A pharmaceutical composition of the invention comprising an insulin derivative, which is A14E, B16H, B25H, B29K(N^{ϵ}-Hexadecandioyl-γGlu), desB30 human insulin (Compound 2).
3. A pharmaceutical composition of the invention comprising an insulin derivative, which is A14E, B16H, B25H, B29K(N^{ϵ}-Eicosanedioyl-γGlu), desB30 human insulin (Compound 3).
4. A pharmaceutical composition of the invention comprising an insulin derivative, which is A14E, B25H, desB27, B29K(N^{ϵ}-Octadecandioyl-γGlu), desB30 human insulin (Compound 4).
5. The pharmaceutical composition of the previous embodiments, wherein the insulin derivative is in the range of from about 3.5 to about 5.0 mM.
6. The pharmaceutical composition of the previous embodiments, wherein the insulin derivative is in the range of from about 4.0 to about 4.5 mM.
7. The pharmaceutical composition of the previous embodiments, wherein the insulin derivative is about 4.2 mM.
8. The pharmaceutical composition of the previous embodiments, comprising of from about 1 to about 2% (weight/weight) of glycerol.
9. The pharmaceutical composition of the previous embodiments, comprising of from about 1.4 to about 1.8% (weight/weight) of glycerol.
10. The pharmaceutical composition of the previous embodiments, comprising about 1.5% or 1.6% (weight/weight) of glycerol.
11. The pharmaceutical composition of the previous embodiments, comprising of from about 45 to about 75 mM of phenol.
12. The pharmaceutical composition of previous embodiments, comprising of from about 50 to about 70 mM of phenol.
13. The pharmaceutical composition of previous embodiments, comprising of from about 55 to about 65 mM of phenol.
14. The pharmaceutical composition of the previous embodiments, comprising about 50, 51, 52, 53, 54, 55, 56, 57, 58, 59, 60, 61, 62, 63, 64, 65, 66, 67, 68, 69, or 70 mM of phenol.
15. The pharmaceutical composition of the previous embodiments, comprising of from about 0 to about 19 mM of m-cresol.
16. The pharmaceutical composition of the previous embodiments, comprising 0 mM, 1 mM, 2 mM, 3 mM, 4 mM of m-cresol.
17. The pharmaceutical composition of the previous embodiments, comprising of from about 0 to about 15 mM of m-cresol.
18. The pharmaceutical composition of the previous embodiments, comprising about 5 mM, 6 mM, 7 mM, 8 mM, 9 mM, 10 mM, 11 mM, 12 mM, 13 mM, 14 mM, 15 mM, 16 mM, 17 mM, 18 mM or 19 mM of m-cresol.
19. The pharmaceutical composition of the previous embodiments, comprising of from about 1.5 to about 2.5 moles of zinc ions per six moles of insulin derivative.
20. The pharmaceutical composition of the previous embodiments, comprising of from about 2.0 to about 2.4 moles of zinc ions per six moles of insulin derivative.
21. The pharmaceutical composition of the previous embodiments, comprising about 2.0 or 2.1 moles of zinc ions per six moles of insulin derivative.
22. The pharmaceutical composition embodiments, comprising about 2.2 or 2.3 moles of zinc ions per six moles of insulin derivative.
23. The pharmaceutical composition of the previous embodiments, comprising about 2.4 or 2.5 moles of zinc ions per six moles of insulin derivative.
24. The pharmaceutical composition of the previous embodiments, comprising less than about 75 mM of sodium chloride.
25. The pharmaceutical composition of the previous embodiments, comprising of from about 5 to about 50 mM of sodium chloride.
26. The pharmaceutical composition of the previous embodiments, comprising of from about 10 to about 25 mM of sodium chloride.
27. The pharmaceutical composition of the previous embodiments, comprising of from about 15 to about 25 mM of sodium chloride.
28. The pharmaceutical composition of the previous embodiments, comprising about 20 mM, 50 mM or 75 mM of sodium chloride.
29. The pharmaceutical composition of the previous embodiments, which has a pH value in the range of from 7.2 to 8.0.
30. The pharmaceutical composition of the previous embodiments, which has a pH value in the range of from 7.2 to 7.6.
31. The pharmaceutical composition of the previous embodiments, which has a pH value about 7.2, 7.3, 7.4, 7.5, 7.6, 7.7, 7.8, or 7.9.
32. The pharmaceutical composition of the previous embodiments, comprising of from about 4.0 to about 4.5 mM of insulin derivative; of from about 1 to about 2% (weight/weight) of glycerol;
   of from about 50 to about 70 mM of phenol;
   of from about 0 to about 15 mM of m-cresol;
   of from about 2.0 to about 2.5 moles of zinc ions per six moles of insulin derivative;
   less than about 50 mM of sodium chloride; and
   is having a pH value in the range of from 7.2 to 7.6.
33. The pharmaceutical composition of the previous embodiments, comprising
   of about 4.2 mM of insulin derivative;
   of about 1.5% (weight/weight) of glycerol;
   of about 60 mM of phenol;
   of about 0 mM of m-cresol;
   of about 2.0 moles of zinc ions per six moles of insulin derivative;
   of about 20 mM of sodium chloride; and
   is having a pH value of about 7.4.
34. The pharmaceutical composition of previous embodiments, comprising
   of about 4.2 mM of insulin derivative;
   of about 1.5% (weight/weight) of glycerol;
   of about 60 mM of phenol;
   of about 10 mM of m-cresol;
   of about 2.0 moles of zinc ions per six moles of insulin derivative;
   of about 20 mM of sodium chloride; and
   is having a pH value of about 7.4.
35. The pharmaceutical composition of the previous embodiments, comprising
   of about 4.2 mM of insulin derivative;
   of about 1.5% (weight/weight) of glycerol;
   of about 60 mM of phenol;
   of about 0 mM of m-cresol;
   of about 2.2 moles of zinc ions per six moles of insulin derivative;
   of about 20 mM of sodium chloride; and
   is having a pH value of about 7.4.
36. The pharmaceutical composition previous embodiments, comprising
   of about 4.2 mM of insulin derivative;
   of about 1.5% (weight/weight) of glycerol;
   of about 60 mM of phenol;
   of about 10 mM of m-cresol;
   of about 2.2 moles of zinc ions per six moles of insulin derivative;
   of about 20 mM of sodium chloride; and
   is having a pH value of about 7.4.
37. The pharmaceutical composition of previous embodiments, comprising
   of about 4.2 mM of insulin derivative;
   of about 1.5% (weight/weight) of glycerol;
   of about 60 mM of phenol;
   of about 0 mM of m-cresol;
   of about 2.4 moles of zinc ions per six moles of insulin derivative;
   of about 20 mM of sodium chloride; and
   is having a pH value of about 7.4.
38. The pharmaceutical composition of the previous embodiments, comprising
   of about 4.2 mM of insulin derivative;
   of about 1.5% (weight/weight) of glycerol;
   of about 60 mM of phenol;
   of about 10 mM of m-cresol;
   of about 2.4 moles of zinc ions per six moles of insulin derivative;
   of about 20 mM of sodium chloride; and
   is having a pH value of about 7.4.
39. The pharmaceutical composition of the previous embodiments, comprising of about 4.2 mM of insulin derivative; of from about 1 to about 2% (weight/weight) of glycerol; of from about 45 to about 75 mM of phenol; of from about 0 to about 15 mM of m-cresol; of from about 1.5 to about 2.5 moles of zinc ions per six moles of said insulin derivative; not more than about 50 mM of sodium chloride; and having a pH value in the range of from 7.2 to 8.0.
40. The pharmaceutical composition of the previous embodiments, comprising about 0 mM of m-cresol.
41. The pharmaceutical composition of the previous embodiments, comprising of from about 5 to about 10 mM of m-cresol.
42. The pharmaceutical composition of the previous embodiments, comprising about 10 mM of m-cresol.
43. The pharmaceutical composition according to the previous embodiments, further comprising semaglutide.
44. A pharmaceutical composition comprising A14E, B16H, B25H, B29K((N^{ϵ}-Eicosanedioyl-γGlu-[2-(2-{2-[2-(2-aminoethoxy)ethoxy]acetylamino}ethoxy)ethoxy]-acetyl)), desB30 human insulin (Compound 1); and semaglutide; and further comprising of from about 1 to about 2% (weight/weight) of glycerol; of from about 45 to about 75 mM of phenol; of from 0-15 mM of m-cresol; of from about 1.5 to about 2.5 moles of zinc ions per six moles of said insulin derivative; not more than about 25 mM of sodium chloride; and having a pH value in the range of from 7.2 to 8.0.
45. The pharmaceutical composition according to the previous embodiments, comprising from about 0.20 to about 0.70 mM semaglutide.
46. The pharmaceutical composition according to the previous embodiments, comprising from about 0.30 to about 0.70 mM semaglutide.
47. The pharmaceutical composition according to the previous embodiments, comprising from about 3.5 mM to about 5.0 mM A14E, B16H, B25H, B29K((N^{ϵ}-Eicosanedioyl-γGlu-[2-(2-{2-[2-(2-aminoethoxy)ethoxy]acetylamino}ethoxy)ethoxy]-acetyl)), desB30 human insulin (Compound 1).
48. The pharmaceutical composition according to the previous embodiments, comprising from about 0.30 to about 0.70 mM semaglutide, and from about 3.5 mM to about 5.0 mM A14E, B16H, B25H, B29K((N^{ϵ}-Eicosanedioyl-γGlu-[2-(2-{2-[2-(2-aminoethoxy)ethoxy]acetylamino}ethoxy)ethoxy]acetyl)), desB30 human insulin (Compound 1).
49. The pharmaceutical composition according to the previous embodiments, comprising about 0.30 mM semaglutide, and from about 3.5 mM to about 5.0 mM A14E, B16H, B25H, B29K((N^{ϵ}-Eicosanedioyl-γGlu-[2-(2-{2-[2-(2-aminoethoxy)ethoxy]acetyl-amino}ethoxy)ethoxy]acetyl)), desB30 human insulin (Compound 1).
50. The pharmaceutical composition according to the previous embodiments, comprising about 0.40 mM semaglutide, and 4.2 mM A14E, B16H, B25H, B29K((N^{ϵ}-Eicosanedioyl-γGlu-[2-(2-{2-[2-(2-aminoethoxy)ethoxy]acetylamino}ethoxy)ethoxy]-acetyl)), desB30 human insulin (Compound 1).
51. The pharmaceutical composition according to the previous embodiments, comprising about 0.49 mM or 0.50 mM semaglutide, and 4.2 mM A14E, B16H, B25H, B29K((N^{ϵ}-Eicosanedioyl-γGlu-[2-(2-{2-[2-(2-aminoethoxy)ethoxy]acetylamino}ethoxy)-ethoxy]acetyl)), desB30 human insulin (Compound 1).
52. The pharmaceutical composition according to the previous embodiments, comprising about 0.60 mM semaglutide, and 4.2 mM A14E, B16H, B25H, B29K((N^{ϵ}-Eicosanedioyl-γGlu-[2-(2-{2-[2-(2-aminoethoxy) ethoxy] acetylamino} ethoxy) ethoxy]-acetyl)), desB30 human insulin (Compound 1).
53. A pharmaceutical (coformulation) composition comprising A14E, B16H, B25H, B29K((N^{ϵ}-Eicosanedioyl-γGlu-[2-(2-{2-[2-(2-aminoethoxy)ethoxy]acetylamino}-ethoxy)ethoxy]acetyl)), desB30 human insulin (Compound 1); and semaglutide; and further comprising about 1.5% (weight/weight) of glycerol; of about 60 mM of phenol; about 0 mM of m-cresol; about 2.2 moles of zinc ions per six moles of said insulin derivative; about 20 mM of sodium chloride; and having a pH value of about 7.4.
54. A pharmaceutical (coformulation) composition comprising A14E, B16H, B25H, B29K((N^{ϵ}-Eicosanedioyl-γGlu-[2-(2-{2-[2-(2-aminoethoxy)ethoxy]acetylamino}-ethoxy)ethoxy]acetyl)), desB30 human insulin (Compound 1); and semaglutide; and further comprising about 1.5% (weight/weight) of glycerol; of about 60 mM of phenol; about 10 mM of m-cresol; about 2.2 moles of zinc ions per six moles of said insulin derivative; about 20 mM of sodium chloride; and having a pH value of about 7.4.
55. The pharmaceutical composition of the previous embodiments, for administration to a subject in need hereof at intervals less frequent than once-daily (i.e. at intervals longer than 24 hours), during a period of time of at least 3 months, at least 6 months, or of at least 1 year.
56. The pharmaceutical composition of the previous embodiments, for administration to a subject in need for administration to the subject with a frequency in the range of from every 2^{nd} day to every 11^{th} day, on average.
57. The pharmaceutical composition of the previous embodiments, for administration to a subject in need for administration to the subject with a frequency in the range of from every 3^{rd} day to every 10^{th} day, on average.
58. The pharmaceutical composition of the previous embodiments, for administration to a subject in need for administration to the subject with a frequency in the range of from every 4^{th} day to every 9^{th} day, on average.
59. The pharmaceutical composition of the previous embodiments, for administration to a subject in need for administration to the subject with a frequency in the range of from every 5^{th} day to every 8^{th} day, on average.
60. The pharmaceutical composition of the previous embodiments, for administration to a subject in need for administration to the subject with a frequency in the range of from every 6^{th} day to every 7^{th} day, on average.
61. The pharmaceutical composition of the previous embodiments, for administration to a subject in need for administration to the subject once a week, i.e. on every 7th day, on average, during a period of time of at least 3 months, at least 6 months, or of at least 1 year.
62. A method for making injectable pharmaceutical composition comprises:
(i) Preparing a solution by dissolving A14E, B16H, B25H, B29K((N^{ϵ}-Eicosanedioyl-γGlu-[2-(2-{2-[2-(2-aminoethoxy)ethoxy]acetylamino}ethoxy)ethoxy]-acetyl)), desB30 human insulin in water
   (ii) Preparing a solution by dissolving preservatives and isotonicity agents in water
   (iii) Preparing a solution by dissolving zinc ions in water
   (iv) Mixing solution a) and solution b)
   (v) Adding solution c) to solution a+b
   (vi) Dissolving semaglutide in the combined solution a+b+c
   (vii) Adjusting the pH of mixture f) to the desired pH, followed by a sterile filtration.

Any combination of two or more of the embodiments described herein is considered within the scope of the present invention.

### Biological Activity

In another aspect the invention provides pharmaceutical compositions useful as medicaments for the treatment of metabolic diseases, disorders or conditions, and, in particular, diseases, disorders or conditions relating to diabetes.

In one embodiment, the pharmaceutical composition of the invention is for use in the treatment or alleviation of a disease, disorder or condition relating to diabetes, Type 1 diabetes, Type 2 diabetes, impaired glucose tolerance, hyperglycemia, dyslipidemia, obesity, or metabolic syndrome (metabolic syndrome X, insulin resistance syndrome).

In another embodiment, the pharmaceutical composition of the invention is for use in the treatment or alleviation of a disease, disorder or condition relating to diabetes, and in particular Type 1 diabetes, or Type 2 diabetes.

The actual dosage depends on the nature and severity of the disease being treated, and is within the discretion of the physician, and may be varied by titration of the dosage to the particular circumstances of this invention to produce the desired therapeutic effect.

### BRIEF DESCRIPTION OF THE DRAWINGS

The present invention is further illustrated by reference to the accompanying drawings, in which:

FIG. 1A, FIG. 1B and FIG. 1C show oligomerisation of Compound 1 mono Formulations A, B, and C at simulated injection site conditions:

FIG. 1A: Apparent average hydrodynamic radius (rH) [nm] measured by DLS;

FIG. 1B: Apparent average molecular weight measured by CG-MALS;

FIG. 1C: Apparent average sedimentation coefficient(S) measured by AUC; White bars: Compound 1, Formulation C with 2.2 Zn++ per six insulins (mol:mol); Grey bars: Compound 1, Formulation B with 2.4 Zn++ per six insulins (mol:mol);

Black bars: Compound 1, Formulation A containing 4.5 Zn++ per six insulins (mol:mol);

FIG. 2A and FIG. 2B show oligomerisation of Compound 1 mono Formulations 01-06 at simulated injection site conditions:

FIG. 2A: Apparent average hydrodynamic radius (Rh, avg) [nm] measured by DLS;

FIG. 2B: Apparent average sedimentation coefficient (S*) measured by AUC

FIG. 3A-FIG. 3D show apparent dynamic viscosity [cP] (FIG. 3A, and FIG. 3C) and specific viscosity [ηspec] (FIG. 3B, FIG. 3D) of different buffer-exchanged formulations of Compound 1 and interstitial fluid buffer (ISF), as a function of temperature [° C.];

FIG. 3A and FIG. 3B: Formulation A; Formulation B; Formulation C; ISF buffer;

FIG. 3C and FIG. 3D: Formulation 02; Formulation 03; Formulation 04; ISF buffer;

FIG. 4A and FIG. 4B show conformational state of Compound 1 in formulations:

FIG. 4A: near-UV CD [Δϵ251 nm (M−1 cm−1)] showing conformational changes (T-state; mixed TR state; R-state) as a function of Zn++ per six insulins (mol:mol) for Formulations 01-06;

FIG. 4B: near-UV CD [Δϵ251 nm (M−1 cm−1)] showing conformational changes (T-state; mixed TR state; R-state) as a function of Zn++ per six insulins (mol:mol) for Formulations B1-B6, D1-D7, and human insulin formulations with various zinc level:

Open circles: Human insulin (600 nmol/ml human insulin, 30 mM phenol, 150 mM NaCl, pH 7.4);

Black squares: Compound 1 formulated with 25 mM phenol, 25 mM m-cresol and 20 mM NaCl;

Grey circles: Compound 1 formulated with 60 mM phenol, 10 mM m-cresol and 20 mM NaCl;

FIG. 5A and FIG. 5B show oligomer distribution of Compound 1 in formulations by SEC:

FIG. 5A shows native SEC chromatogram of Formulations 01, 02, 03, 04, 05, and 06;

FIG. 5B shows native SEC chromatogram of formulations A and B1-B6;

FIG. 6A and FIG. 6B show SAXS scattering data [Intensity (a.u.) vs. s (Å−1)] of Compound 1 and human insulin in the formulated state:

FIG. 6A: Scattering curves of Compound 1 Formulation A shown in black and of human insulin shown in grey (Compound 1, 4.2 mM, 4.5 Zn/hexamer; Human insulin 0.6 mM, 2.2 Zn/hexamer);

FIG. 6B: Scattering curves of Compound 1 Formulation C shown in black and of human insulin shown in grey (Compound 1, 4.2 mM, 2.2 Zn/hexamer; Human insulin 0.6 mM, 2.2 Zn/hexamer (R6-hexamer));

FIG. 7A and FIG. 7B show purity of Compound 1 in formulations:

FIG. 7A shows purity (% of total peptide) at 30° C. storage [Time Point (Month)] for Compound 1 Formulation A (black line), Formulation B (grey line) and Formulation C (dotted line);

FIG. 7B shows purity (% of total peptide) at 37° C. storage [Time Point (weeks)] for Compound 1 for Formulations 01, 04, 05 and 06;

White circles: Formulation 01, Black circles: Formulation 04, White squares: Formulation 05, Black squares: Formulation 06;

FIG. 8 shows purity of Compound 1 (% of total Compound 1) at 37° C. storage [Time Point (weeks)] in combo formulations with semaglutide:

White circles: combo-Formulation I, Black circles: combo-Formulation II, White triangles: combo-Formulation III, Black triangles: combo-Formulation IV, White squares: combo-Formulation V, Black squares: combo-Formulation VI;

FIG. 9A and FIG. 9B show oligomerisation of combo formulations at simulated injection site conditions:

FIG. 9A: Apparent average hydrodynamic radius (rH) [nm] measured by DLS;

FIG. 9B: shows the oligomer size of combo formulations after buffer exchanged as observed by AUC (S*).

### EXAMPLES

The invention is further illustrated with reference to the following examples, which are not intended to be in any way limiting to the scope of the invention as claimed.

### Example 1

### Improved Biophysical Properties at Simulated Injection Site Conditions

### Protocol

The API of the formulations is A14E, B16H, B25H, B29K((N^{ϵ}-Eicosanedioyl-γGlu-[2-(2-{2-[2-(2-aminoethoxy)ethoxy]acetylamino}ethoxy)ethoxy]acetyl)), desB30 human insulin (Compound 1), obtained as described in e.g. WO 2009/115469, see Example 33.

Oligomerisation of Compound 1 was determined at simulated injection site conditions. Simulated injection site conditions were achieved by depletion of phenol and/or metacresol, respectively, and buffer exchange into simulated interstitial fluid buffer. This procedure virtually removed all phenolic ligands but maintained the zinc/insulin ratio of the parent formulation.

Formulations are buffer-exchanged into interstitial fluid (ISF-) buffer via PD-MidiTrap G-25 column according to the manufacturers protocol.

The columns are equilibrated with the target buffer by washing the column with a sufficient volume of target buffer, and the formulation is applied in a suitable volume and eluted with target buffer. The ISF buffer consists of 140 mM NaCl, 4 mM KCl, 1 mM MgSO4, 2 mM CaCl2, 10 mM Hepes, pH 7.4.

Unless otherwise stated, the procedure was the following:
Buffer exchange at 22° C.;
Subsequent incubation at 37° C. for 14-18 hours; and
Subsequent measurement at 22° C. (except otherwise stated).

Typically measurements were conducted about 1 h after the termination of the 37° C. incubation. If this is not possible, samples are stored at 22° C. until measurement. Unless otherwise stated, samples are measured un-diluted. It should be noted that the oligomer size resulting from the described phenol depletion procedure is process dependent, and for a given formulation it may vary with factors such as time, temperature, and batch of column. Therefore it is desirable that measurements for a given set of formulations be compared within the same experiment and not across experiments. Therefore, for the same reference formulation was tested together with various formulation of the invention in different experiments. For example, Formulation A vs. Formulation B and C; Formulation 01 vs. Formulations 02-06.

Apparent average hydrodynamic radius (rH) was measured by Dynamic Light Scattering (DLS), using a DynaPro PR™ (Wyatt technology, Santa Barbara, CA, USA). Before analysis samples were centrifuged for 5 minutes at 1200 rpm to remove any dust particles in the solution. Measurements were conducted at 25° C. using 40 acquisitions and 2 sec acquisition time.

Apparent average molecular weight (kDa) measured by Composition-Gradient, Multi-Angle Static Light Scattering (CG-MALS) using a system consisting of a Wyatt

Technology Corporation (WTC) Calypso II titrator unit coupled to a WTC DAWN8+ light scattering detector (operating at 664 nm) and a WTC Optilab T-rEX refractometer (operating at 660 nm) at 25° C. The samples were filtered through a 0.45 μm filter followed by a 0.22 μm filter prior to measurement.

Sedimentation velocity (SV) experiments were performed with an XL-I Analytical

Ultracentrifuge (BeckmanCoulter, Brea, CA) in 12-mm or 3-mm double-sector centrepieces capped with sapphire windows. Samples were spun at 40000 rpm and 20° C. until sedimentation was completed and monitored with the interference optics of the instrument. Sedimentation Coefficient Distributions (SCD) were calculated with SedFit, version 11.8 (www.analyticalultracentrifugation.com) using the c(s) model with a grid of 100 s-values over a range sufficient to describe all sedimenting material, as judged by the rmsd and the residual run pattern. The frictional ratio f/fo was treated as a variable to be optimized during fitting (P Schuck, M A Perugini, N R Gonzales, G J Howlett and D Schubert: Size-distribution analysis of proteins by analytical ultracentrifugation: strategies and application to model systems; (Biophys. J. 2002 82:1096). Average sedimentation coefficient values were obtained via integration of the resulting c(s)-distributions.

Temperature-dependent dynamic viscosities were measured with a Lovis2000 rolling-ball type viscosimeter (Anton Paar, Graz, Austria). The temperature was decreased from 40° C. to 4° C. in 2° C. steps, allowing 5 minutes of temperature equilibration between steps. The density of the buffer was simultaneously measured with a DMA5000 densitometer, also from Anton Paar.

Three mono formulations, i.e. Formulation A representative of the prior art (see e.g. WO 2013/153000), and Formulations B, C representative of the invention (60 mM phenol/10 mM m-cresol), were prepared.

TABLE 1A
Comparative mono formulations A, B, and C

| | Formulation A | Formulation B | Formulation C |
|---|---|---|---|
| | Representative of | Representative of | Representative of the |
| Ingredient | the prior art | the invention | invention |
| Compound 1 | 4200 nmol/ml | 4200 nmol/ml | 4200 nmol/ml |
| | (4.2 mM) | (4.2 mM) | (4.2 mM) |
| Zinc (as zinc | 206 μg/ml | 110 μg/ml | 101 μg/ml |
| acetate) | (~4.5 | (~2.4 | (~2.2 Zn⁺⁺/hexamer) |
| | Zn⁺⁺/hexamer) | Zn⁺⁺/hexamer) | |
| Glycerol | 16 mg/ml (1.6%) | 15 mg/ml (1.5%) | 15 mg/ml (1.5%) |
| Phenol | 2.35 mg/ml (25 | 5.65 mg/ml (60 | 5.65 mg/ml (60 mM) |
| | mM) | mM) | |
| Meta-cresol | 2.70 mg/ml (25 | 1.08 mg/ml (10 | 1.08 mg/ml (10 mM) |
| | mM) | mM) | |
| Sodium | 1.17 mg/ml (20 | 1.17 mg/ml (20 | 1.17 mg/ml (20 mM) |
| chloride | mM) | mM) | |
| pH | 7.4 | 7.4 | 7.4 |

Another six mono formulations, i.e. Formulation 01 (same as Formulation A), and Formulations 02, 03, 04, 05 and 06 representative of the invention, were made (Table 1B). The zinc content has been varied from 4.5 Zn⁺⁺/six insulins to 2.4, 2.2, and 2.0 Zn++/six insulins. In addition preservative systems of either 25/25 mM phenol/m-cresol or 60/0 mM phenol/m-cresol have been tested.

TABLE 1B
Comparative formulations

| | Formulation | | | | | |
|---|---|---|---|---|---|---|
| Ingredient | 01* | 02 | 03 | 04 | 05 | 06 |
| Compound 1 | 4.2 | 4.2 | 4.2 | 4.2 | 4.2 | 4.2 |
| (mM) | | | | | | |
| Zn(acetate) 2 | 206 | 206 | 101 | 101 | 110 | 92 |
| (μg/ml) | | | | | | |
| n Zn/6 insulin | ~4.5 | ~4.5 | ~2.2 | ~2.2 | ~2.4 | ~2.0 |
| (mol/mol) | | | | | | |
| Phenol | 25 mM | 60 mM | 25 mM | 60 mM | 60 mM | 60 mM |
| m-cresol | 25 mM | 0 | 25 mM | 0 | 0 | 0 |
| glycerol | 1.6% | 1.5% | 1.6% | 1.5% | 1.5% | 1.5% |

| | |
|---|---|
| Vehicle (all | 20 mM NaCl |
| formulations) | pH 7.4 |

*Same as formulation A

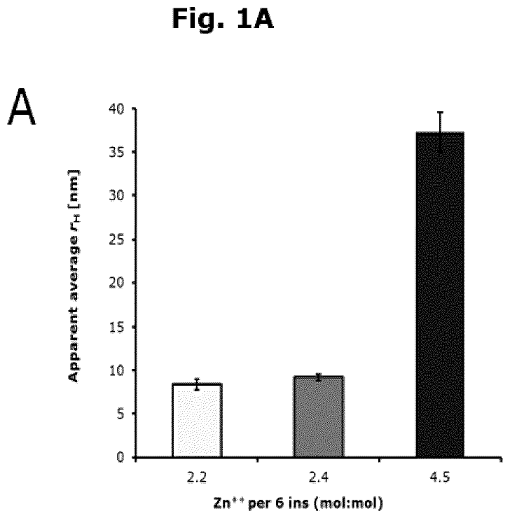

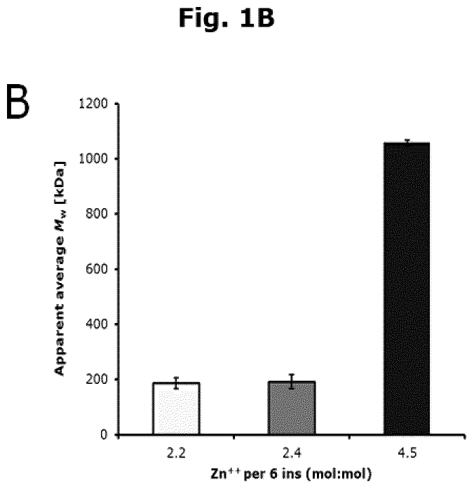

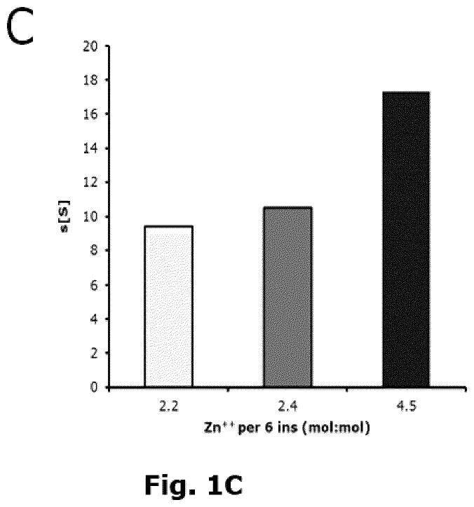

Improved oligomer size of buffer exchanged mono-formulations having 60 mM phenol and 10 m-cresol (Table 2A; FIGS. 1A, 1B, and 1C)

TABLE 2A
Oligomer size at simulatied injection site

| | A | B | C |
|---|---|---|---|
| Rh(avg) [nm] | 37.31 | 9.21 | 8.37 |
| S*(avg) [S] | 17.32 | 10.53 | 9.41 |
| Mw (avg) [kDa] | 1059.8 | 190.5 | 184.7 |

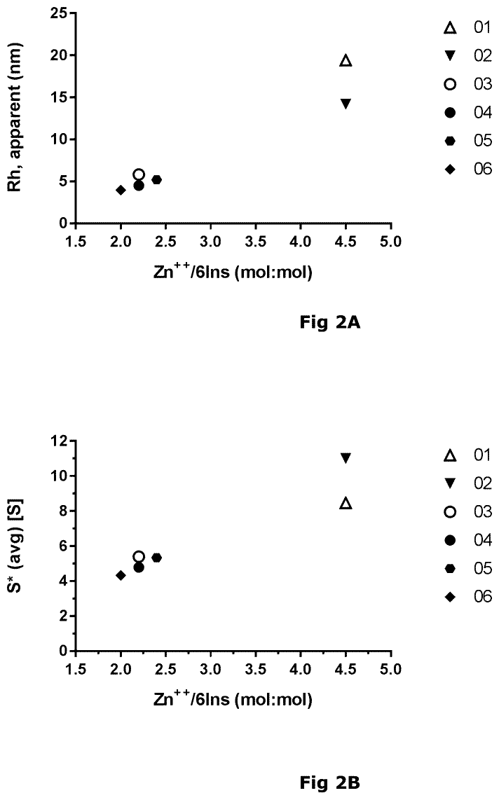

Improved oligomer size of buffer exchanged mono-formulations having 60 mM phenol and 0 m-cresol (Table 2B; FIGS. 2A and 2B)

TABLE 2B
Oligomer size at simulatied injection site

| | 01 | 02 | 03 | 04 | 05 | 06 |
|---|---|---|---|---|---|---|
| Rh(avg) [nm] | 19.42 | 14.20 | 5.83 | 4.51 | 5.20 | 3.98 |
| S*(avg) [S] | 8.46 | 11.00 | 5.39 | 4.78 | 5.33 | 4.32 |

### Conclusion

The zinc content was decreased from 4.5 Zn⁺⁺/six insulins in Formulation A to 2.4 and 2.2 Zn⁺⁺/six insulins in formulations B and C (with increased phenol and decreased metacresol) respectively. This decrease in zinc was accompanied by a reduction of oligomer size (see FIGS. 1A, 1B and 1C) as determined at simulated injection site conditions by the method described above.

Results from formulation 01-06 further confirm that the oligomer size is reduced when Zn is reduced from 4.5 Zn⁺⁺/six insulins to 2.2±0.2 Zn⁺⁺/six insulins. For 2.2±0.2 Zn⁺⁺/six insulins the oligomer size is further reduced when phenol/cresol is reduced from 25 mM/25 mM to 60 mM/0 mM. See FIGS. 2A and 2B.

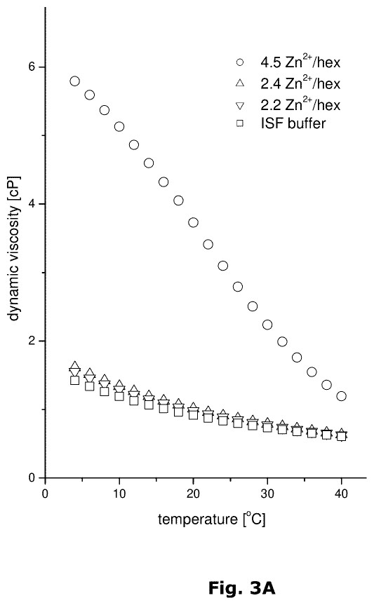

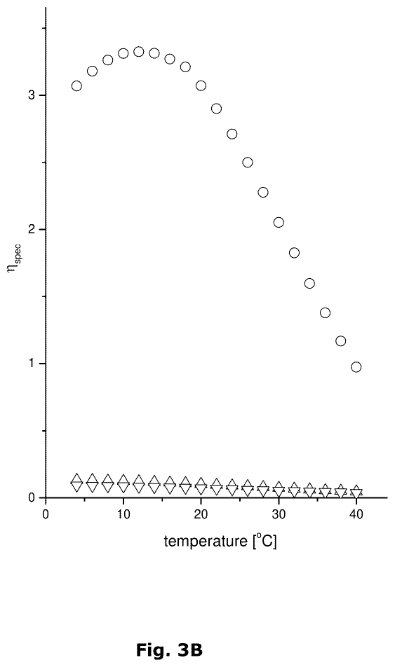

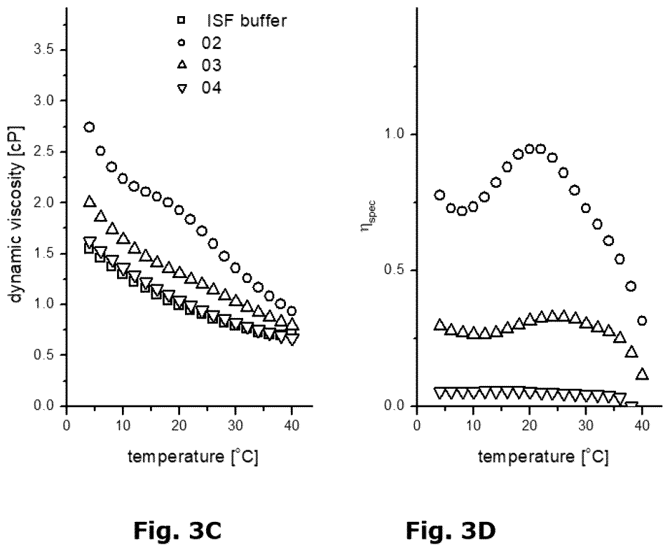

Improved viscosity of buffer exchanged mono-formulations having 60 mM phenol and 10 mM m-cresol (see Table 3A; FIGS. 3A and 3B)

TABLE 3A
Viscosity of buffer-exchanged Formulations A, B, C

| | Dynamic viscosity (cP) | | | | Specific viscosity | | |
|---|---|---|---|---|---|---|---|
| temp | Formulation | | | | Formulation | | |
| (C.) | A | B | C | ISF buffer | A | B | C |
| 40 | 1.194 | 0.633 | 0.628 | 0.605 | 0.97355 | 0.04628 | 0.03802 |
| 38 | 1.359 | 0.659 | 0.653 | 0.627 | 1.16746 | 0.05104 | 0.04147 |
| 36 | 1.548 | 0.687 | 0.68 | 0.651 | 1.37788 | 0.0553 | 0.04455 |
| 34 | 1.758 | 0.718 | 0.709 | 0.677 | 1.59675 | 0.06056 | 0.04727 |
| 32 | 1.989 | 0.751 | 0.74 | 0.704 | 1.82528 | 0.06676 | 0.05114 |
| 30 | 2.237 | 0.787 | 0.773 | 0.733 | 2.05184 | 0.07367 | 0.05457 |
| 28 | 2.506 | 0.825 | 0.809 | 0.765 | 2.27582 | 0.07843 | 0.05752 |
| 26 | 2.792 | 0.866 | 0.847 | 0.798 | 2.49875 | 0.08521 | 0.0614 |
| 24 | 3.098 | 0.91 | 0.889 | 0.835 | 2.71018 | 0.08982 | 0.06467 |
| 22 | 3.409 | 0.958 | 0.934 | 0.874 | 2.90046 | 0.09611 | 0.06865 |
| 20 | 3.729 | 1.01 | 0.982 | 0.916 | 3.07096 | 0.10262 | 0.07205 |
| 18 | 4.051 | 1.066 | 1.035 | 0.962 | 3.21102 | 0.10811 | 0.07588 |
| 16 | 4.321 | 1.126 | 1.092 | 1.012 | 3.26976 | 0.11265 | 0.07905 |
| 14 | 4.597 | 1.191 | 1.153 | 1.066 | 3.31238 | 0.11726 | 0.08161 |
| 12 | 4.865 | 1.262 | 1.22 | 1.125 | 3.32444 | 0.12178 | 0.08444 |
| 10 | 5.131 | 1.338 | 1.293 | 1.19 | 3.31176 | 0.12437 | 0.08655 |
| 8 | 5.369 | 1.421 | 1.372 | 1.26 | 3.26111 | 0.12778 | 0.08889 |
| 6 | 5.593 | 1.514 | 1.458 | 1.338 | 3.18012 | 0.13154 | 0.08969 |
| 4 | 5.795 | 1.612 | 1.556 | 1.424 | 3.06952 | 0.13202 | 0.0927 |

Improved viscosity of buffer exchanged mono-formulations having 60 mM phenol and 0 m-cresol (see Table 3B; FIGS. 3C and 3D)

TABLE 38
Viscosity of buffer exchanged Formulations 02, 03 and 04

| | Dynamic viscosity (cP) | | | | Specific viscosity | | |
|---|---|---|---|---|---|---|---|
| | Formulation | | | | Formulation | | |
| | ISF | _02 | _03 | _04 | _02 | _03 | _04 |
| 3.99 | 1.5447 | 2.7441 | 1.9996 | 1.6278 | 0.77646 | 0.29449 | 0.0538 |
| 6 | 1.4513 | 2.5087 | 1.8529 | 1.5296 | 0.72859 | 0.27672 | 0.05395 |
| 8 | 1.3668 | 2.3492 | 1.7355 | 1.4414 | 0.71876 | 0.26975 | 0.05458 |
| 10 | 1.2917 | 2.2389 | 1.6335 | 1.3615 | 0.7333 | 0.26461 | 0.05404 |
| 12 | 1.2209 | 2.1624 | 1.5448 | 1.2894 | 0.77115 | 0.2653 | 0.05611 |
| 14 | 1.1555 | 2.1078 | 1.4678 | 1.2215 | 0.82415 | 0.27027 | 0.05712 |
| 16 | 1.095 | 2.06 | 1.4063 | 1.1575 | 0.88128 | 0.28429 | 0.05708 |
| 18 | 1.04 | 2.0041 | 1.3516 | 1.0984 | 0.92702 | 0.29962 | 0.05615 |
| 20 | 0.9893 | 1.927 | 1.3014 | 1.043 | 0.94784 | 0.31548 | 0.05428 |
| 22 | 0.9423 | 1.8351 | 1.247 | 0.9916 | 0.94747 | 0.32336 | 0.05232 |
| 24 | 0.8991 | 1.7221 | 1.1941 | 0.9447 | 0.91536 | 0.32811 | 0.05072 |
| 26 | 0.8588 | 1.597 | 1.1404 | 0.9003 | 0.85957 | 0.3279 | 0.04832 |
| 28 | 0.8216 | 1.4748 | 1.085 | 0.8594 | 0.79503 | 0.32059 | 0.04601 |
| 30 | 0.7869 | 1.3608 | 1.0249 | 0.8219 | 0.72932 | 0.30245 | 0.04448 |
| 32 | 0.7544 | 1.2594 | 0.9713 | 0.7867 | 0.66941 | 0.28751 | 0.04282 |
| 34 | 0.7239 | 1.1654 | 0.9215 | 0.754 | 0.60989 | 0.27297 | 0.04158 |
| 36 | 0.7004 | 1.0795 | 0.8749 | 0.7236 | 0.54126 | 0.24914 | 0.03312 |
| 38 | 0.6951 | 1.0027 | 0.8321 | 0.6948 | 0.44253 | 0.19709 | −4.32E−04 |
| 40 | 0.7107 | 0.9345 | 0.7927 | 0.6682 | 0.3149 | 0.11538 | −0.0598 |

### Conclusion

These experiments show that the viscosity of the formulation at simulated injection site conditions according to this method are highly dependent on the zinc content such that decreasing the zinc ratio leads to lower viscosity.

### Example 2

tₘₐₓ and elimination t_{1/2}

tₘₐₓ represents the time to maximum concentration (maximal plasma exposure), and t_{1/2} represents the elimination half-life in which half of the compound disappears from plasma following administration.

The altered biophysical properties described in Example 1 are consistent with a PK profile exhibiting earlier tₘₐₓ in pigs (see Table 3). This indicates that the residence time of Compound 1 in the sub-cutis is reduced when formulated according to the invention, and in contrast to the same compound being provided in a formulation according to the prior art.

Surprisingly, the reduced zinc level almost has no impact on the duration of action (i.e. elimination t_{1/2} is not affected), which makes the formulation according to the invention and the formulation of the prior art equally suited for once-weekly administration.

TABLE 4

| | | tₘₐₓ | t_{1/2} |
|---|---|---|---|
| Formulation | Species | (hours) | (hours) |
| Compound 1, Formulation A | Pig | 20 | 47 |
| 4.5 zinc/hexamer, 25 mM phenol, 25 | Human | 42 | 185 |
| mM m-cresol | | | |
| Compound 1, Formulation C | Pig | 8 | 45 |
| 2.2 zinc/hexamer, 60 mM phenol, 10 | | | |
| mM m-cresol, 20 mM NaCl | | | |

### Conclusion

These experiments show that the residence time in the sub-cutis (tₘₐₓ) of Compound 1 was significantly reduced as a result of reducing Zn⁺⁺/hexamer. The observation is consistent with data presented in Example 1, which show a reduction of the size of the oligomers formed at simulated injection site conditions.

Thus, when formulated according to the invention, Compound 1 forms smaller oligomers at the site of injection resulting in a PK/PD profile with a shorter residence time in the sub-cutis (decreased tₘₐₓ).

But surprisingly, although the residence time in the sub-cutis (tₘₐₓ) of Compound 1 was significantly reduced, the formations of the invention maintained the same elimination half-life (elimination t_{1/2} is not affected). This means the formulations of the invention could facilitate the long-acting insulin derivatives to more quickly reach to the maximum concentration in circulation, while still maintain the maximum concentration with longer duration.

This unexpected finding also makes it possible to limit the formation of large oligomers at the site of injection, while still preserving a long duration of action commensurate with a once-weekly administration profile. Formation of large oligomers with high viscosity at the site of injection may introduce discomfort upon injection.

### Example 3

### Improved Conformational State in Formulation

With presence of zinc, human insulin exists as hexamers in formulation. Human insulin hexamers can adopt two different conformational states depending on the conformation of the monomers. The eight N-terminal amino acid residues of the insulin monomer B-chain can be in either an extended conformation (T-state) or an a-helical conformation (R-state). In the presence of phenol and NaCl, human insulin adopts the R conformation, which is the favourable conformation with regards to physical and chemical stability (Dunn M F. Zinc-ligand interactions modulate assembly and stability of the insulin hexamer: A review. Biometals 2005; 18; 295-303).

### Protocol

The conformational changes of the insulin hexamer were followed by the 251 nm CD-signature (Krüger P, Gilge G, Cabuk Y and Wollmer A; Biol. Chem. Hoppe-Seyler 1990 371 669-673). Samples were measured using a Jasco 815 instrument and a pathlength of 0.02 cm. Blank titrations were subtracted and the resulting As 251 nm was plotted vs Zn/6Ins (mol/mol).

Conformational State of Mono-Formulations Having 60 mM Phenol/0 m-cresol

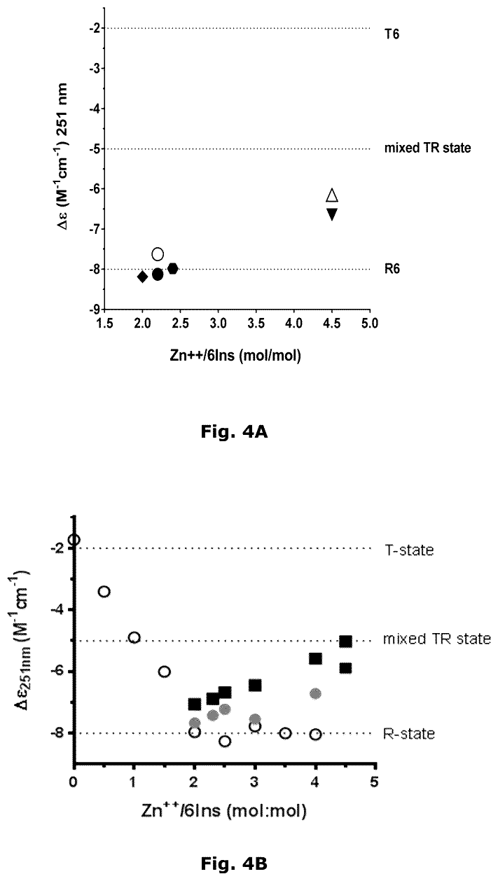

The conformational state of Formulations 01-06 is shown in Table 5A and FIG. 4A.

TABLE 5A
Conformational state propensity in
formulation 01-06 with 60 mM phenol/0 m-cresol

| Formulation | 01 | 02 | 03 | 04 | 05 | 06 |
|---|---|---|---|---|---|---|
| R-state propensity | −6.15 | −6.65 | −7.63 | −8.12 | −7.98 | −8.19 |
| Δε (M-1cm-1) @ 251 nm | | | | | | |

FIG. 4A shows the conformational changes (T-state; mixed TR state; R-state) as a function of Zn⁺⁺ per six insulins (mol:mol) for formulations 01-06.

Conformational State of Mono-Formulations Having 60 mM Phenol and 10 mM m-cresol

Two series of formulations were prepared. All formulations comprised 4.2 mM of Compound 1, 16 mg/ml (1.6%) glycerol, 20 mM NaCl, with pH of 7.4. Series B formulations comprised 60 mM phenol and 10 mM m-cresol, with zinc level varied as 1.5 (B1), 2.0 (B2), 2.3 (B3), 2.5 (B4), 3.0 (B5), and 4.0 (B6) per 6 insulin. Series D comprised 25 mM phenol and 25 mM m-cresol, with zinc level varied as 1.5, 2.0, 2.3, 2.5, 3.0, 4.0 and 4.5 per 6 insulin. Also a series of human insulin formulations were prepared, with various zinc level. The conformational state is shown in Table 5B and FIG. 4B.

TABLE 5B
Conformational state propensity in formulations B1-B6
with 60 mM phenol and 10 m-cresol, and
formulations D1-D7 with 25 mM phenol and 25 m-cresol

| R-state propensity | | |
|---|---|---|
| Δε (M-1 cm-1) | | |
| @251 nm | Series B | Series D |
| Zn/6Ins | 60/10 phe/cre | 25/25 phe/cre |
| *1.5 | −6.83 (B1) | −6.58 (D1) |
| 2.0 | −7.67 (B2) | −7.06 (D2) |
| 2.3 | −7.42 (B3) | −6.88 (D3) |
| 2.5 | −7.23 (B4) | −6.67 (D4) |
| 3.0 | −7.54 (B5) | −6.45 (D5) |
| 4.0 | −6.72 (B6) | −5.58 (D6) |
| 4.5 | n.a. | −5.03 (D7) |

*Not included in FIG. 4B

FIG. 4B shows conformational changes (T-state; mixed TR state; R-state) as a function of Zn++ per six insulins (mol:mol) for Series B and Series D formulations, and human insulin formulations.

### Conclusion

Near-UV CD data show that the T/R conformation of Compound 1 was dependent on the zinc concentration. A decrease in zinc was accompanied by a change in conformational state of Compound 1 in the formulation from a mixed T/R state to the

R-state, which was also accompanied by a higher level of hexamer formed in the formulations (see Example 4 below). The R-state and hexmer level was further enhanced by a change in phenol/meta-cresol ratio from 25/25 mM to 60/10 mM (FIG. 4B).

The data of formulations 01-06 show that R-state of Compound 1 in the formulations is are further enriched by decreasing Zn from 4.5 to 2.2Zn +0.2Zn/6 Compound 1 and by omitting m-cresol (FIG. 4A).

Example 4

### Improved Oligomer Distribution in Formulation

### Protocol

Size Exclusion Chromatography is a sensitive method for quantifying the non-covalent oligomer distribution of insulin formulations. SEC was conducted using a BEH200 column, 1.7 μm 4.6×150 mm with a running buffer consisting of 8.0 mM phenol, 140 mM NaCl, 10 mM Tris-HCl, pH 7.4. Chromatography was conducted at 22 C using 2 μL injection volume and a flow of 0.3 ml/min. As molecular weight standards albumin, a covalent insulin hexamer, and a monomeric insulin were used. Chromatograms were analysed by integration to represent species larger than hexamer (3.0-3.8 min), hexamer (3.8-4.3 min), and species smaller than hexamer (4.3-5.5 min). Please note that exact integration limits for each data set will vary slightly due to variations in column performance.

Small angle X-ray scattering (SAXS) data were collected using a BioSAXS-2000 instrument equipped with a flow cell and a Dectris 100K detector covering a q-range of 0.008-0.661 Å⁻¹. Buffer measurements were subtracted from sample measurements to get the protein scattering profiles. Data from a reference sample of human insulin in a hexameric state with R conformation was collected according to the same procedure, from a sample of 0.6 mM human insulin, 3 Zn⁺⁺/six insulins, 16 mM phenol, 20 mM NaCl and 7 mM phosphate buffer pH 7.4.

Oligomer Distribution for Formulations Comprising 60 mM Phenol and 0 m-cresol

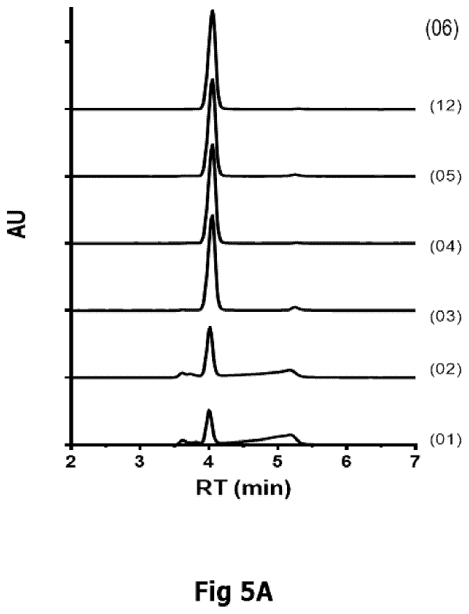

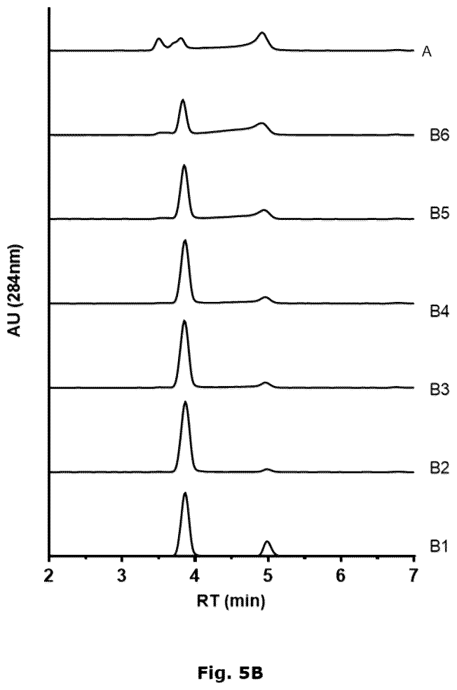

Native SEC chromatogram comparing formulations 01, 02, 03, 04, 05, and 06 has been generated (see Table 6 and FIG. 5A).

TABLE 6
Species distribution as obtained by native SEC
by integration of chromatograms

| | | Formulation | | | | | |
|---|---|---|---|---|---|---|---|
| | Peak | 01 | 02 | 03 | 04 | 05 | 06 |
| | assignment | % species in formulation | | | | | |
| (3.0-3.8 | Larger than | 8.8 | 9.4 | 1.1 | 0.6 | 0.7 | 0.2 |
| min) | hexamer | | | | | | |
| (3.8-4.3 | Hexamer | 34.5 | 48.9 | 95.1 | 98.1 | 95.9 | 98.9 |
| min) | | | | | | | |
| (4.3-5.5 | smaller than | 56.8 | 41.7 | 3.8 | 1.3 | 3.5 | 0.9 |
| min) | hexamer | | | | | | |

Oligomer Distribution for Formulations Comprising 60 mM Phenol and 10 mM m-cresol

Native SEC chromatogram comparing formulations B1-B6 and formulation A has been generated (see Table 6 and FIG. 5B). The area under the curve is similar for all chromatograms. Chromatograms were analysed by integration to represent species larger than hexamer (3.0-3.8 min), hexamer (3.8-4.3 min), and species smaller than hexamer (4.3-5.5 min). Please note that exact integration limits for each data set will vary slightly due to variations in column performance.

TABLE 7
Oligomer distribution by SEC

| | Formulation | | | | | | |
|---|---|---|---|---|---|---|---|
| Peak | B1 | B2 | B3 | B4 | B5 | B6 | A |
| assignment | % species in formulation | | | | | | |
| Larger than | 0.2 | 0.5 | 0.8 | 0.7 | 2.2 | 4.9 | 18.2 |
| hexamer | | | | | | | |
| Hexamer | 80.3 | 94.8 | 86.7 | 82.1 | 65.5 | 41.8 | 22.0 |
| smaller than | 19.5 | 4.8 | 12.5 | 17.2 | 32.3 | 53.3 | 59.8 |
| hexamer | | | | | | | |

### Conclusion

SEC data of Formulations 01-06 (Table 6, FIG. 5A) show that the oligomer distribution of Compound 1 Formulation 01 is characterised by broad bands with no clear dominant oligomer species. The oligomer distribution of Compound 1 in Formulations 03-06 is narrower compared to Formulations 01 and 02. The retention time of the main peak Formulations 03-06 is consistent with a hexamer and the small peak with a retention time of 5 minutes is consistent with a monomer or dimer.

SEC data of Formulations B1-B6 and A (Table 7, FIG. 5B) also show that with 2.0-2.5 zinc/6 insulins, the hexamer peak is enriched, compared to 4.0 or 4.5 Zn.

Therefore, the hexamer peak is enriched at low zinc (e.g., 2.0Zn to 2.5Zn) compared to high zinc (e.g. 4.0 or 4.5Zn). For both 4.5Zn and 2.2Zn the hexamer is enriched at 60 mM/0 or 10 mM phenol/m-cresol relative to 25/25 mM phenol/m-cresol. When formulated according to the invention the hexamer content is increased and the oligomerisation becomes more well-defined.

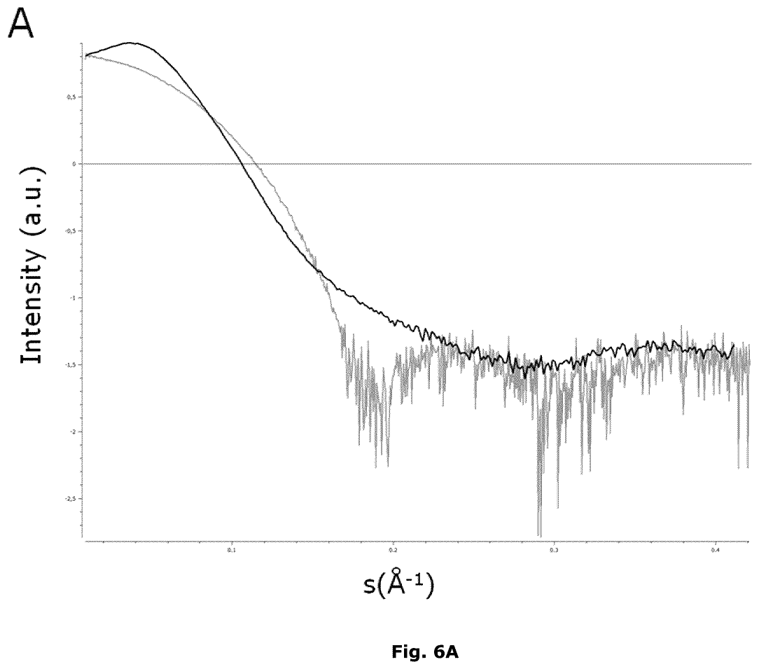

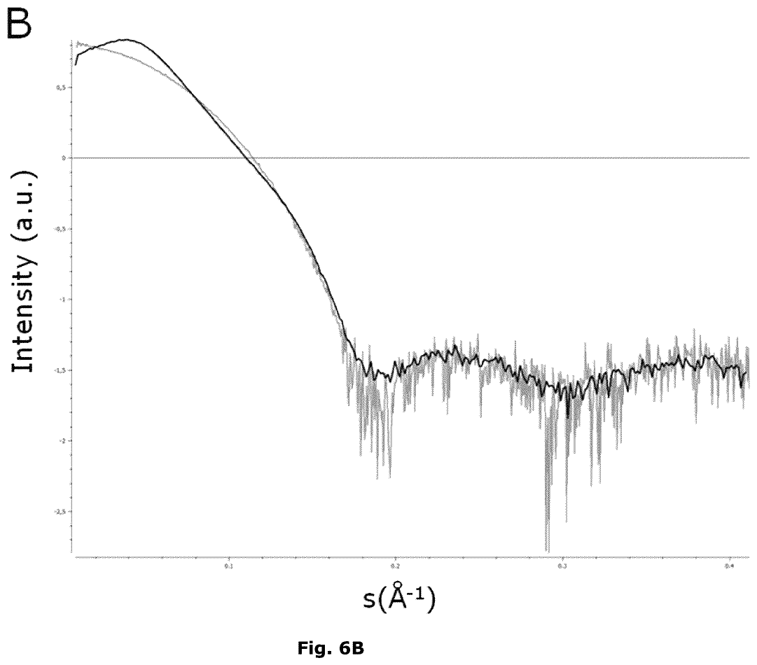

SAXS data also confirm that the oligomerisation pattern of Compound 1 Formulation A with 4.5 Zn⁺⁺/hexamer does not resemble the classical human insulin hexamer while a hexamer-based structure of the Compound 1 is dominant in Formulation C (FIG. 6). When formulated according to the invention (Formulation C) the oligomerisation pattern becomes more well-defined and consistent with a hexamer based structure similar to human insulin.

### Example 5

### Improved Chemical Stability and Physical Stability

Experimental results in this example showed that both chemical stability and physical stability of Compound 1 are improved of the new formulations compared with the reference Formulation A. In particular, with high level of phenol and low level of m-cresol, both chemical stability and physical stability of Compound 1 increases when zinc concentration decreases.

### Protocol

Purity was determined by reversed phase ultra-high performance liquid chromatography (RP-UHPLC) where the samples were analysed using a Acquity CSH

Fluoro Phenyl, 130 Å, 1.7 μm, 2.1×150 mm column, gradient elution by acetonitrile in a mobile phase of acetonitrile and phosphoric buffer in water with subsequent UV detection (215 nm) under a flow of 0.30 ml/min with a sample injection volume of 2-3 μl. Purity was evaluated as the area of the main peak divided by the area of all peaks×100%.

Chemical Stability of Compound 1 Formulation with 60 mM Phenol and 10 mM m-cresol

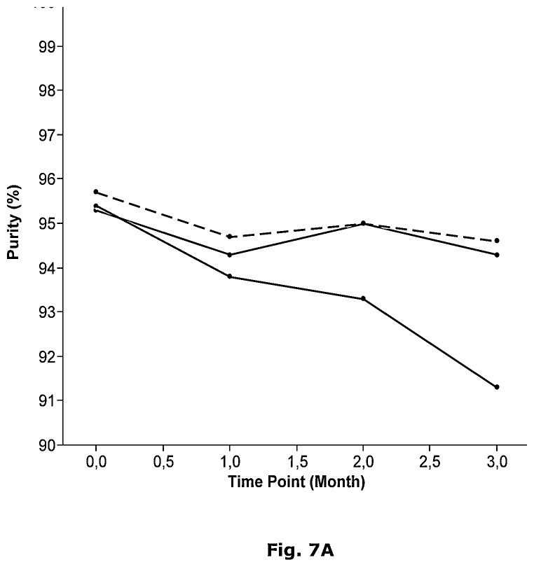

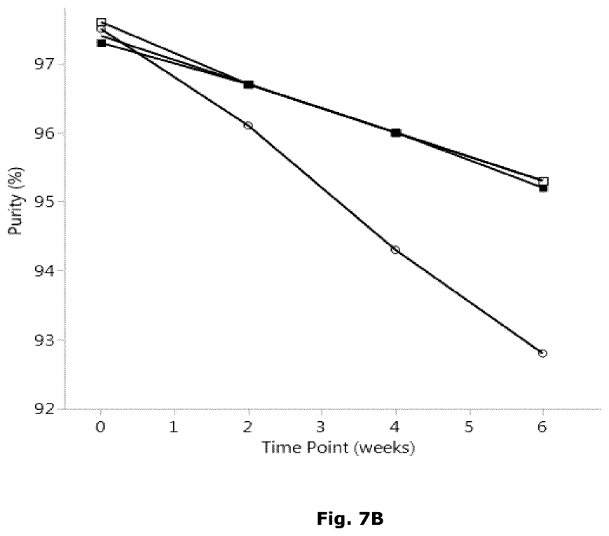

The stability measured as % purity of total peptide with RP-UHPLC shows an significantly increased stability of Compound 1 in Formulation B and Formulation C, which comprised 60 mM phenol and 10 mM m-cresol, compared to Compound 1 Formulation A, which comprised 25 mM phenol and 25 mM m-cresol. See Table 8 and FIG. 7A.

TABLE 8
% Purity of Compound 1

| | Storage time at 30° C. (Months) | | | |
|---|---|---|---|---|
| Formulation | 0 | 1 | 2 | 3 |
| A | 95.4 | 93.8 | 93.3 | 91.3 |
| B | 95.3 | 94.3 | 95.0 | 94.3 |
| C | 95.7 | 94.7 | 95.0 | 94.6 |

Chemical Stability of Compound 1 Formulations with 60 mM Phenol and 0 m-cresol

The stability of formulations presented in table 9 measured as % purity of total peptide with RP-UHPLC further confirms that the chemical stability of Compound 1 increases as a function of zinc concentration in formulations with relatively high level of phenol and low level of m-cresol. The results show an increased stability of Compound 1 when zinc was decreased from 4.5 Zn⁺⁺/six insulins to 2.4, 2.2 and 2.0 Zn++/six insulins in formulations with a change in preservative system from 25/25 mM phenol/m-cresol to 60 mM phenol and 0 m-cresol. See Table 9 and FIG. 7B.

TABLE 9
% Purity of Compound 1

| | Storage time at 37° C. (weeks) | | | |
|---|---|---|---|---|
| Formulation | 0 | 2 | 4 | 6 |
| 01 | 97.5 | 96.1 | 94.3 | 92.8 |
| 04 | 97.4 | 96.7 | 96.0 | 95.3 |
| 05 | 97.6 | 96.7 | 96.0 | 95.3 |
| 06 | 97.3 | 96.7 | 96.0 | 95.2 |

### Conclusion

When formulated according to the invention (see Formulations B, C, 04, 05, and 06; Tables 8 and 9, and FIG. 7A and FIG. 7B) the chemical stability increases compared to prior art (Formulations A and Formulation 01).

### Physical Stability

### Protocol:

The Compound 1 formulations were tested in a 96-well microtiter plate with 4 replica of 200 μl. To 1.0 ml from each formulation, ThT (thioflavin T) was added to 1 μM. Thioflavin T (ThT) assay for propensity to form amyloid fibrils was performed on Thermo Fluoroskan, 960 rpm shaking, 37° C., for 45 hours. ThT emission was scanned before and after assay. The lag time until on-set of ThT fluorescence emission is a measurement of physical stability. Lag times were determined from fluorescence curves averaged over 4 replica. A longer lag-time is indicative of higher physical stability.

It should be noted that the ThT results obtained from the described protocol may vary between experiments. Therefore it is desirable that measurements for a given set of formulations be compared within the same experiment and not across experiments. In this Example, the same reference formulation was tested together with various formulations of the invention in different experiments. For example, Formulation A vs. Formulation C; Formulation 01 vs. Formulations 04-06.

The lag times are shown in Table 10 below. Formulation A or Formulation 01 as reference formulation for comparison.

The results are show in Table 10A and Table 10B.

Physical stability of Compound 1 in formulations with 60 mM phenol and 10 m-cresol

TABLE 10A
Tht lag times (1)

| | Formulation C | Formulation A |
|---|---|---|
| Lag time (hrs) | 11.3 | 10.0 |

TABLE 10B
Tht lag times (2)

| | 01 | 04 | 05 | 06 |
|---|---|---|---|---|
| Lag time | 9.7 | 29.3 | 13.3 | >45 |
| (hrs) | | | | |

### Conclusion

The lag-times obtained in the ThT assay indicates that the physical stability of Compound 1 is improved in formulations with low level of zinc, high level of phenol, and low level of m-cresol. The data showed that when decreasing zinc from 4.5Zn/6Ins to 2.2±0.2Zn/6Ins and concomitant increasing phenol from 25 mM to 60 mM and decreasing m-cresol from 25 mM to 10 mM lead to longer lag time and thus improved physical stability. With m-cresol being removed, the physical stability of Compound 1 was further improved in formulations with low zinc level (see Table 10B).

### Example 6

### Combo-Formulations and the Chemical and Physical Stability Thereof

Compound 1 may be co-formulated together with the once-weekly GLP-1 analogue semaglutide for a fixed-ratio combination.

The following combo-formulations 1 to 6 of Compound 1 and semaglutide were prepared. The mono-formulation of Compound 1 was also prepared as mono-formulation reference 1. The intended target values are shown in Table 11, below.

TABLE 11
Combo-formulations of Compound 1 and semaglutide

| | Mono | Combo | Combo | Combo | Combo | Combo | Combo |
|---|---|---|---|---|---|---|---|
| | Reference 1 | 1 | 2 | 3 | 4 | 5 | 6 |
| Compound 1 | 4.2 | 4.2 | 4.2 | 4.2 | 4.2 | 4.2 | 4.2 |
| (mM) | | | | | | | |
| Semaglutide | n.a. | 0.49/2.0 | 0.4/1.6 | 0.6/2.4 | 0.3/1.25 | 0.49/2.0 | 0.49/2.0 |
| (mM)/(mg/ml) | | | | | | | |
| Zn(acetate)₂ | 1.54 | 1.54 | 1.54 | 1.54 | 1.54 | 1.4 | 1.75 |
| (mM) | | | | | | | |
| n Zn/6 insulin | −2.2 | −2.2 | −2.2 | −2.2 | −2.2 | −2.0 | −2.5 |

| | |
|---|---|
| Vehicle (all | 60 mM phenol |
| formulations) | 10 mM m-cresol |
| | 1.5% glycerol |
| | 20 mM NaCl |
| | pH 7.4 |

The concentrations of Compound 1 and semaglutide in the produced formulations were measured using RP-HPLC and reference materials. These concentrations are stated in Table 12A and 12B, below.

TABLE 12A
Measured concentrations of Compound 1 and semaglutide

| | Mono | | | | |
|---|---|---|---|---|---|
| | Reference-1 | Combo 1 | Combo 2 | Combo 3 | Combo 4 |
| Compound 1 | 4.1 | 4.1 | 4.1 | 4.1 | 4.1 |
| (mM) | | | | | |
| Measured | | | | | |
| Semaglutide | n.a. | 0.5 | 0.4 | 0.6 | 0.3 |
| (mM) | | | | | |
| Measured | | | | | |

TABLE 12B
Measured concentrations of Compound 1 and semaglutide
(a separate batch from Table 12A)

| | Mono | | |
|---|---|---|---|
| | reference-1 | Combo 5 | Combo 6 |
| Compound 1 | 4.2 | 4.3 | 4.3 |
| (mM) | | | |
| Measured | | | |
| Semaglutide | n.a. | 0.49 | 0.49 |
| (mM) | | | |
| Measured | | | |

The measured concentrations thus deviated less than 3% from the intended target values.

The following combo-formulations I-VI of Compound 1 and semaglutide were also prepared later. The mono-formulation of Compound 1 was prepared as mono-formulation reference 2. The intended target values are shown in Table 13, below.

TABLE 13

| | Mono | Combo | Combo | Combo | Combo | Combo | Combo |
|---|---|---|---|---|---|---|---|
| | Reference 2 | I | II | III | IV | V | VI |
| Compound 1 | 4.2 | 4.2 | 4.2 | 4.2 | 4.2 | 4.2 | 4.2 |
| (mM) | | | | | | | |
| Semaglutide | n.a. | 0.49/2.0 | 0.49/2.0 | 0.3/1.25 | 0.49/2.0 | 0.49/2.0 | 0.3/1.25 |
| (mM)/(mg/ml) | | | | | | | |
| Zn(acetate)₂ | 101 | 206 | 101 | 101 | 101 | 110 | 110 |
| (μg/ml) | | | | | | | |
| n Zn/6 insulin | ~2.2 | ~4.5 | ~2.2 | ~2.2 | ~2.2 | ~2.4 | ~2.4 |
| Phenol | 60 mM | 25 mM | 60 mM | 60 mM | 25 mM | 60 mM | 60 mM |
| m-cresol | 0 | 25 mM | 0 | 0 | 25 mM | 0 | 0 |

| | |
|---|---|
| Vehicle (all | 1.5% glycerol |
| formulations) | 20 mM NaCl |
| | pH 7.4 |

### Physical Stability

### Protocol:

The formulations were tested in a 96-well microtiter plate with 8 replica of 200 μl. To 1.0 ml from each formulation, ThT (thioflavin T) was added to 1 μM. Thioflavin T (ThT) assay for propensity to form amyloid fibrils was performed on Thermo Fluoroskan, 960 rpm shaking, 37° C., for 45 hours. ThT emission was scanned before and after assay. The lag time until on-set of ThT fluorescence emission (formation of amyloid fibrils) is a measurement of physical stability. Lag times were determined from fluorescence curves averaged over 8 replica. Lag times were tested twice for combo formulations 1-4 and combo formulations 5 and 6, respectively; and each with mono formulation tested as a reference, to make the results comparable. A longer lag-time is indicative of higher physical stability.

The Compound 1 mono-formulation reference and the combo-formulations were tested in a 96-well microtiter plate Thioflavin T (ThT) assay for propensity to form amyloid fibrils.

The lag times are shown in Table 14A, 14B, 14C below.

TABLE 14A
Combo-formulation lag times

| | Mono | | | | |
|---|---|---|---|---|---|
| | Reference 1 | Combo 1 | Combo 2 | Combo 3 | Combo 4 |
| Lag time | 11.0 | 19.3 | 18.0 | 21.6 | 23 |
| (hrs) | | | | | |

TABLE 14B
Combo-formulation lag times

| | Mono | | |
|---|---|---|---|
| | Reference 1 | Combo 5 | Combo 6 |
| Lag time | 9.9 | 20.6 | 45 |
| (hrs) | | | |

TABLE 14C
Combo-formulation lag times

| | Mono | Combo | Combo | Combo | Combo | Combo | Combo |
|---|---|---|---|---|---|---|---|
| | reference 2 | I | II | III | IV | V | VI |
| Lag | 29.3 | 44.3 | 45 | 45 | 28.6 | 45 | 45 |
| time | | | | | | | |
| (hrs) | | | | | | | |

### Conclusion:

The ThT assay indicated that without increasing zinc level, combo formulations of Compound 1 and semaglutide did not compromise the physical stability of Compound 1 compared to that of the Compound 1 mono-formulation. In fact, the lag times of the combo-formulations were much longer than that of the Compound 1 mono-formulation, showing the co-formulating of Compound 1 with semaglutide in fact stabilizes the formulation towards the unwanted amyloid fibril formation. Compared with combo-formulation of other long acting insulin derivative and GLP-1 derivative (e.g. degludec and liraglutide), this finding is unexpected and surprising.

Table 14C results show that lowering the level of m-cresol can further improve the physical stability of the combo-formulation of Compound 1 and semaglutide; and increasing the level of phenol also improves the physical stability of the combo-formulation of Compound 1 and semaglutide.

Table 14C results also show that when co-formulating Compound 1 with semaglutide in a formulation according to the invention, the physical stability of Compound 1 increases, compared to using prior art formulation (Formulation I) for combo-formulation of Compound 1 and semaglutide.

### Chemical Stability

### Protocol

Purity was determined by reversed phase ultra-high performance liquid chromatography (RP-UHPLC) where the samples were analysed using a Acquity CSH Fluoro Phenyl, 130 Å, 1.7 um, 2.1×150mm column, gradient elution by acetonitrile in a mobile phase of acetonitrile and phosphoric buffer in water with subsequent UV detection (215 nm) under a flow of 0.30 ml/min with a sample injection volume of 2-3 μl. Purity was evaluated as the area of the main peak divided by the area of all peaks x 100%.

The stability of formulations presented in table 15 measured as % purity of total Compound 1 with RP-UHPLC.

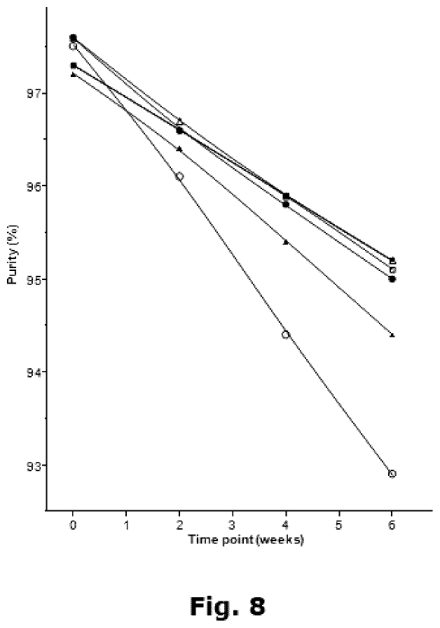

The results confirm an increased chemical stability of Compound 1 in the combo-formulations when zinc is decreased from 4.5 Zn++/six insulins to 2.4, 2.2 and 2.0 Zn++/six insulins (Table 15, FIG. 8). A change in preservative system from 25/25 mM phenol/m-cresol to 60 mM phenol and 0 m-cresol results in an additional improvement in chemical stability of Compound 1 in combo-formulation.

TABLE 15
Purity of Compound 1 in combo-formulation

| Combo- | Purity with storage time at 37° C. (weeks) | | | |
|---|---|---|---|---|
| Formulation | 0 | 2 | 4 | 6 |
| I | 97.5% | 96.1% | 94.4% | 92.9% |
| II | 97.6% | 96.6% | 95.8% | 95.0% |
| III | 97.6% | 96.7% | 95.9% | 95.2% |
| IV | 97.2% | 96.4% | 95.4% | 94.4% |
| V | 97.3% | 96.6% | 95.9% | 95.1% |
| VI | 97.3% | 96.6% | 95.9% | 95.2% |

### Conclusion

When co-formulating Compound 1 with semaglutide in a formulation according to the invention, the chemical stability of Compound 1 increases, compared to using prior art formulation (Formulation I) for combo-formulation of Compound 1 and semaglutide.

### Example 7

PK Properties of Co-Formulations with Semaglutide in LYD Pig PK Model

Of the formulations produced in Example 6, Compound 1 reference mono formulation and Combo 1 and combo 2 were characterized in the LYD pig PK animal model. It is important that the PK parameters tₘₐₓ and t_{1/2} of Compound 1, as well as mean residence time (MRT) of Compound 1 were not significantly altered upon co-formulation with semaglutide.

A cross-over study with 16 animals (n=8 for each formulation) was conducted.

TABLE 16
PK parameters of Compound 1

| | Mono | | | |
|---|---|---|---|---|
| | reference 1 | Combo 1 | Combo 2 | |
| tₘₐₓ (hr) | 13.0 ± 7.0 | 9.0 ± 2.0 | | 8.0 ± 2.0 |
| t_{1/2} (hr) | 48.0 ± 4.1 | 48.6 ± 3.2 | | 47.9 ± 5.3 |
| MRT (hr) | 71 ± 6 | 69.0 ± 4.0 | | 68.0 ± 4.0 |

Average values±standard deviation are shown.

The PK parameters for Compound 1 when co-formulated with semaglutide in Combo 1 and Combo 2 were not significantly changed compared to Compound 1 administrated as a mono-formulation. The tₘₐₓ values were slightly lower for the co-formulations, but with the standard deviation for the Compound 1 reference the values are overlapping. The t_{1/2} and MRT were very similar for Compound 1 in the co-formulations compared to the reference mono-formulation. In conclusion the PK properties of Compound 1 were not significantly impacted by the co-formulation with semaglutide.

### Example 8

### Improved Oligomer Size of Buffer Exchanged Co-Formulations 60/0

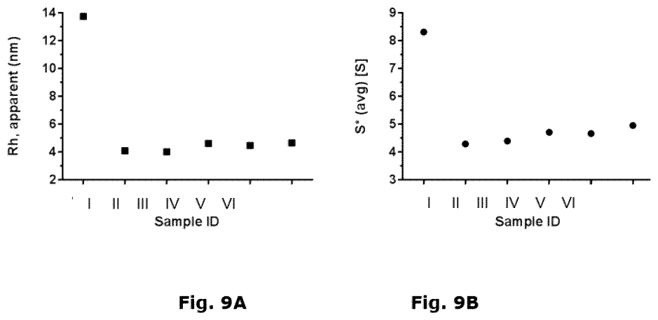

The oligomerisation of combo formulations I-VI formed at simulated injection site conditions of Compound 1 was determined according to the protocol described in Example 1. The results are shown in Table 17 and FIG. 9A and FIG. 9B.

TABLE 17
Oligomer size of combo-formulations

| | Combo | Combo | Combo | Combo | Combo | Combo |
|---|---|---|---|---|---|---|
| | I | II | III | IV | V | VI |
| Rh(avg) [nm] | 13.7 | 4.1 | 4.0 | 4.6 | 4.5 | 4.7 |
| S*(avg) [S] | 8.32 | 4.30 | 4.40 | 4.72 | 4.67 | 4.96 |

### Conclusion

These experiments show that the size of the oligomers formed at simulated injection site conditions are highly dependent on the zinc content.

The average size of oligomers formed from combo-formulations at simulated injection site conditions is significantly reduced in formulations with low level of zinc (e.g., 2.4 and 2.2 Zn++/six insulins) compared to formulations with high level zinc (e.g. 4.5 Zn++/six insulin). Increasing the level of phenol and decreasing the level of m-cresol further reduce the average size of oligomers formed from combo-formulations at simulated injection site conditions.

1. A pharmaceutical composition comprising an insulin derivative selected from the group consisting of
A14E, B16H, B25H, B29K((N^{ϵ}-Eicosanedioyl-γGlu-[2-(2-{2-[2-(2-aminoethoxy)ethoxy]-acetylamino}ethoxy)ethoxy]acetyl)), desB30 human insulin (Compound 1);
A14E, B16H, B25H, B29K(N^{ϵ}-Hexadecandioyl-γGlu), desB30 human insulin (Compound 2);
A14E, B16H, B25H, B29K(N^{ϵ}-Eicosanedioyl-γGlu), desB30 human insulin (Compound 3); and
A14E, B25H, desB27, B29K(N^{ϵ}-Octadecandioyl-γGlu), desB30 human insulin (Compound 4); and further comprising
of from about 1 to about 2% (weight/weight) of glycerol; of from about 45 to about 75 mM of phenol; of from about 0 to about 19 mM of m-cresol; of from about 1.5 to about 2.5 moles of zinc ions per six moles of said insulin derivative; not more than about 75 mM of sodium chloride; and having a pH value in the range of from 7.2 to 8.0.

2. The pharmaceutical composition according to claim 1, wherein the insulin derivative is A14E, B16H, B25H, B29K((N^{ϵ}-Eicosanedioyl-γGlu-[2-(2-{2-[2-(2-aminoethoxy)ethoxy]-acetylamino}ethoxy)ethoxy]acetyl)), desB30 human insulin (Compound 1).

3. The pharmaceutical composition according to claim 1, wherein the amount of insulin derivative is in the range of from about 3.5 to about 5.0 mM.

4. The pharmaceutical composition according to claim 1, comprising of from about 1 to about 2% (weight/weight) of glycerol.

5. The pharmaceutical composition according to claim 1, comprising of from about 45 to about 75 mM of phenol, such as from about 55 mM to about 65 mM of phenol; or comprising of 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, or 70 mM of phenol.

6. The pharmaceutical composition according to claim 1, comprising of from about 0 to about 19 mM of m-cresol, such as from about 0 mM to about 15 mM of m-cresol; or comprising of 0 mM, 1 mM, 2 mM, 3 mM, 4 mM, 5 mM, 6 mM, 7 mM, 8 mM, 9 mM, 10 mM, 11 mM, 12 mM, 13 mM, 14 mM, or 15 mM of m-cresol.

7. The pharmaceutical formulation according to claim 1, comprising of from about 1.5 to about 2.5 moles of zinc ions per six moles of insulin derivative.

8. The pharmaceutical composition according to claim 1, comprising less than about 25 mM of sodium chloride.

9. The pharmaceutical composition according to claim 1, having a pH value in the range of from 7.2 to 8.0.

10. The pharmaceutical composition according to claim 1, comprising
of from about 4.0 to about 4.5 mM of insulin derivative;
of from about 1 to about 2% (weight/weight) of glycerol;
of from about 50 to about 70 mM of phenol;
of from about 0 to about 15 mM of m-cresol; of from about 2.0 to about 2.5 moles of zinc ions per six moles of insulin derivative;
no more than about 25 mM of sodium chloride; and
having a pH value in the range of from 7.2 to 7.6.

11. The pharmaceutical composition according to claim 1, comprising
of about 4.2 mM of insulin derivative;
of about 1.5% (weight/weight) of glycerol;
of about 60 mM of phenol;
of about 0 mM of m-cresol;
of about 2.2 moles of zinc ions per six moles of insulin derivative;
of about 20 mM of sodium chloride; and
having a pH value of about 7.4.

12. The pharmaceutical composition according to claim 1, comprising
of about 4.2 mM of insulin derivative;
of about 1.5% (weight/weight) of glycerol;
of about 60 mM of phenol;
of about 10 mM of m-cresol;
of about 2.2 moles of zinc ions per six moles of insulin derivative;
of about 20 mM of sodium chloride; and
having a pH value of about 7.4.

13. The pharmaceutical composition according to claim 1, further comprising semaglutide.

14. The pharmaceutical composition according to claim 13, wherein the amount of semaglutide is in the range of from about 0.30 to about 0.70 mM.

15. The pharmaceutical composition according to claim 2, wherein the amount of insulin derivative is in the range of from about 3.5 to about 5.0 mM.

16. The pharmaceutical composition according to claim 2, comprising of from about 1 to about 2% (weight/weight) of glycerol.

17. The pharmaceutical composition according to claim 2, comprising of from about 45 to about 75 mM of phenol, such as from about 55 mM to about 65 mM of phenol; or comprising of 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, or 70 mM of phenol.

18. The pharmaceutical composition according to claim 2, comprising of from about 0 to about 19 mM of m-cresol, such as from about 0 mM to about 15 mM of m-cresol; or comprising of 0 mM, 1 mM, 2 mM, 3 mM, 4 mM, 5 mM, 6 mM, 7 mM, 8 mM, 9 mM, 10 mM, 11 mM, 12 mM, 13 mM, 14 mM, or 15 mM of m-cresol.

19. The pharmaceutical formulation according to claim 2, comprising of from about 1.5 to about 2.5 moles of zinc ions per six moles of insulin derivative.

20. The pharmaceutical composition according to claim 2, comprising less than about 25 mM of sodium chloride.

21. The pharmaceutical composition according to claim 2, having a pH value in the range of from 7.2 to 8.0.

22. The pharmaceutical composition according to claim 2, comprising
of from about 4.0 to about 4.5 mM of insulin derivative;
of from about 1 to about 2% (weight/weight) of glycerol;
of from about 50 to about 70 mM of phenol;
of from about 0 to about 15 mM of m-cresol; of from about 2.0 to about 2.5 moles of zinc ions per six moles of insulin derivative;
no more than about 25 mM of sodium chloride; and
having a pH value in the range of from 7.2 to 7.6.

23. The pharmaceutical composition according to claim 2, comprising
of about 4.2 mM of insulin derivative;
of about 1.5% (weight/weight) of glycerol;
of about 60 mM of phenol;
of about 0 mM of m-cresol;
of about 2.2 moles of zinc ions per six moles of insulin derivative;
of about 20 mM of sodium chloride; and
having a pH value of about 7.4.

24. The pharmaceutical composition according to claim 2, comprising
of about 4.2 mM of insulin derivative;
of about 1.5% (weight/weight) of glycerol;
of about 60 mM of phenol;
of about 10 mM of m-cresol;
of about 2.2 moles of zinc ions per six moles of insulin derivative;
of about 20 mM of sodium chloride; and
having a pH value of about 7.4.

25. The pharmaceutical composition according to claim 2, further comprising semaglutide.

26. The pharmaceutical composition according to claim 25, wherein the amount of semaglutide is in the range of from about 0.30 to about 0.70 mM.

27. A method of treating a metabolic disorder, comprising administering the pharmaceutical composition according to claim 1 to a subject in need thereof.
